(19) United States
(12) Patent Application Publication
(10) Pub. No.: US 2005/0019788 A1
(43) Pub. Date: Jan. 27, 2005

(54) Genetic markers for skatole metabolism

(75) Inventors: E. James Squires, Guelph (CA); Zhihong Lin, Guelph (CA); Yanping Lou, Guelph (CA)

Correspondence Address: MCKEE, VOORHEES & SEASE, P.L.C., 801 GRAND AVENUE, SUITE 3200, DES MOINES, IA, 50309-2721, US

(73) Assignee: UNIVERSITY OF GUELPH, Guelph (CA)

(21) Appl. No.: 10/769,507

(22) Filed: Jan. 30, 2004

Related U.S. Application Data
(63) Continuation-in-part of application No. 10/024,628, filed on Nov. 23, 2001.
Application No. 10/024,628 is a continuation-in-part of application No. 09/288,037, filed on Apr. 8, 1999, now abandoned.
(60) Provisional application No. 60/081,037, filed on Apr. 8, 1998.

(51) Int. Cl. C12Q 1/68

(52) U.S. Cl. 435/6

(57) ABSTRACT

Disclosed herein are novel alleles characterized by polymorphisms in sulfotransferase genes. The alleles may be used to genetically type animals for sulfotransferase activity. In a preferred embodiment, the alleles may be used as markers for boar taint in pigs. Methods for identifying such markers, and methods of screening animals to determine those more likely to produce desired characteristics and preferably selecting those animals for future breeding purposes are also disclosed.

### CROSS-REFERENCE TO RELATED APPLICATIONS

This application claims benefit from U.S. application Ser. No. 10,024,628 filed Nov. 23, 2001, which is a continuation-in-part of U.S. application Ser. No. 09/288,037 filed Apr. 8, 1999 (now abandoned), which is a non-provisional of U.S. Applicant No. 60/081,037 filed Apr. 8, 1998, all of which are incorporated herein by reference.

### FIELD OF THE INVENTION

This invention relates generally to the detection of genetic differences among animals. More particularly, the invention relates to polymorphisms that affect enzyme efficiency and are indicative of heritable phenotypes associated with boar taint in porcine. Methods and compositions for use of these genetic differences in genotyping of animals and selection are also disclosed as well as novel sequences.

### BACKGROUND OF THE INVENTION

Male pigs that are raised for meat production are usually castrated shortly after birth to prevent the development of off-odors and off flavors (boar taint) in the carcass. Boar taint is primarily due to high levels of either the 16-androstene steroids (especially 5.alpha. (-androst-16-en-3-one)) or skatole in the fat. Skatole is produced by bacteria in the hindgut which degrade tryptophan that is available from undigested feed or from the turnover of cells lining the gut of the pig (Jensen and Jensen, 1995). Skatole is absorbed from the gut and metabolized primarily in the liver (Jensen and Jensen, 1995). High levels of skatole can accumulate in the fat, particularly in male pig, and the presence of a recessive gene Ska.sup.1, which results in decreased metabolism and clearance of skatole has been proposed (Lundstrom et al., 1994; Friis, 1995). Skatole metabolism has been studied extensively in ruminants (Smith, et al., 1993), where it can be produced in large amounts by ruminal bacteria and results in toxic effects on the lungs (reviewed in Yost, 1989). The metabolic pathways involving skatole have not been well described in pigs. In particular, the reasons why only some intact male pigs have high concentrations of skatole in the fat are not clear. Environmental and dietary factors are important (Kjeldsen, 1993; Hansen et al., 1995) but do not sufficiently explain the reasons for the variation in fat skatole concentrations in pigs. Claus et al. (1994) proposed high fat skatole concentrations are a result of an increased intestinal skatole production due to the action of androgens and glucocorticoids. Lundstrom et al. (1994) reported a genetic influence on the concentrations of skatole in the fat, which may be due to the genetic control of the enzymatic clearance of skatole. The liver is the primary site of metabolism of skatole and liver enzymatic activities could be the controlling factor of skatole deposition in the fat. Baebuttedk et al. (1995) described several liver metabolites of skatole found in blood and urine with the major being MII and MIII. MII, which is a sulfate conjugate of 6-hydroxyskatole (pro-MII), was only found in high concentrations in plasma of pigs which were able to rapidly clear skatole from the body, whereas high MIII concentrations were related to slow clearance of skatole. Thus the capability of synthesis of MII could be a major step in a rapid metabolic clearance of skatole resulting in low concentrations of skatole in fat and consequently low levels of boar taint.

In view of the foregoing, further work is needed to fully understand the metabolism of skatole in pig liver and to identify the key enzymes involved. Understanding the biochemical events involved in skatole metabolism can lead to novel strategies for treating, reducing or preventing boar taint. In addition, polymorphisms in these candidate genes may be useful as possible markers for low boar taint pigs.

### SUMMARY OF THE INVENTION

This invention relates to the discovery of genetic variation associated with quantitative trait loci or linkage equilibrium analysis that may be used to predict phenotypic traits in animals. According to the invention, major affect genes have been identified which are related to phenotypic variation in animals. According to the invention, phenotypic variation in skatole metabolism and concomitant boar taint are correlated to major effect alleles linked to variation in sulfotransferase genes. To the extent that this family of genes are conserved among species and animals, and it is expected that the different alleles disclosed herein will also correlate with variability in these gene(s) in other economic or meat-producing animals such as cattle, sheep, chicken, etc with concomitant effects on sulfotransferase activity related to other traits in lieu of or in addition to boar taint.

To achieve the objects and in accordance with the purpose of the invention, as embodied and broadly described herein, the present invention provides the discovery of alternate genotypes which provide a method for genetically typing animals and screening animals to determine those with favorable allelic forms of genes resulting in skatole enzymes with increased or decreased activity and concomitant effects on reduced boar taint or to select against animals which have alleles indicating less favorable characteristics. As used herein a “favorable” or “desired” or “improved” with respect to a trait means a significant improvement (increase or decrease) in one of any measurable indicia of boar taint or other sulfotransferase-related phenotype above the mean of a given group, species line or population, so that this information can be used in breeding to achieve a uniform population which is optimized for these traits. This may include an increase in some traits or a decrease in others depending on the desired characteristics. Traits may also be observed at the molecular level by assaying for activity of enzymes involved in skatole metabolism.

Methods for assaying for these traits generally comprises the steps 1) obtaining a biological sample from a animal; and 2) analyzing the genomic DNA or protein obtained in 1) to determine which allele(s) is/are present. Haplotype data which allows for a series of linked polymorphisms to be combined in a selection or identification protocol to maximize the benefits of each of these markers may also be used.

Since several of the polymorphisms may involve changes in amino acid composition of the respective protein or will be indicative of the presence of this change, assay methods may even involve ascertaining the amino acid composition of the protein of the major effect genes of the invention. Methods for this type or purification and analysis typically involve isolation of the protein through means including fluorescence tagging with antibodies, separation and purification of the protein (i.e. through reverse phase HPLC system), and use of an automated protein sequencer to identify the amino acid sequence present. Protocols for this assay are standard and known in the art and are disclosed in Ausubel et. al.(eds.), Short Protocols in Molecular Biology Fourth ed. John Wiley and Sons 1999.

In another embodiment, the invention comprises a method for identifying genetic markers for boar taint. Once a major effect gene has been identified, it is expected that other variation present in the same gene, allele or in related family of gene sequences in useful linkage disequilibrium therewith may be used to identify similar effects on these traits. The identification of other such genetic variation, once a major effect gene has been discovered, represents more than routine screening and optimization of parameters well known to those of skill in the art and is intended to be within the scope of this invention.

The following terms are used to describe the sequence relationships between two or more nucleic acids or polynucleotides: (a) “reference sequence”. (b) “comparison window”. (c) “sequence identity”. (d) “percentage of sequence identity”. and (e) “substantial identity”.

(a) As used herein, “reference sequence” is a defined sequence used as a basis for sequence comparison. In this case the Reference sequences. A reference sequence may be a subset or the entirety of a specified sequence; for example, as a segment of a full-length cDNA or gene sequence, or the complete cDNA or gene sequence.

(b) As used herein, “comparison window” includes reference to a contiguous and specified segment of a polynucleotide sequence, wherein the polynucleotide sequence may be compared to a reference sequence and wherein the portion of the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence, a gap penalty is typically introduced and is subtracted from the number of matches.

Methods of alignment of sequences for comparison are well-known in the art. Optimal alignment of sequences for comparison may be conducted by the local homology algorithm of Smith and Waterman, Adv. Appl. Math. 2:482 (1981); by the homology alignment algorithm of Needleman and Wunsch, J. Mol. Biol. 48:443 (1970); by the search for similarity method of Pearson and Lipman, Proc. Natl. Acad. Sci. 85:2444 (1988); by computerized implementations of these algorithms, including, but not limited to: CLUSTAL in the PC/Gene program by Intelligenetics, Mountain View, Calif.; GAP, BESTFIT, BLAST, FASTA, and TFASTA in the Wisconsin Genetics Software Package, Genetics Computer Group (GCG), 575 Science Dr., Madison, Wis., USA; the CLUSTAL program is well described by Higgins and Sharp, Gene 73:237-244 (1988); Higgins and Sharp, CABIOS 5:151-153 (1989); Corpet, et al., Nucleic Acids Research 16:10881-90 (1988); Huang, et al., Computer Applications in the Biosciences 8:155-65 (1992), and Pearson, et al., Methods in Molecular Biology 24:307-331 (1994). The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel, et al., Eds., Greene Publishing and Wiley-Interscience, New York (1995).

Unless otherwise stated, sequence identity/similarity values provided herein refer to the value obtained using the BLAST 2.0 suite of programs using default parameters. Altschul et al., Nucleic Acids Res. 25:3389-3402 (1997). Software for performing BLAST analyses is publicly available, e.g., through the National Center for Biotechnology-Information (http://www.hcbi.nlm.nih.gov/).

This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul, Proc. Natl. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance.

BLAST searches assume that proteins can be modeled as random sequences. However, many real proteins comprise regions of nonrandom sequences which may be homopolymeric tracts, short-period repeats, or regions enriched in one or more amino acids. Such low-complexity regions may be aligned between unrelated proteins even though other regions of the protein are entirely dissimilar. A number of low-complexity filter programs can be employed to reduce such low-complexity alignments. For example, the SEG (Wooten and Federhen, Comput. Chem., 17:149-163 (1993)) and XNU (Claverie and States, Comput. Chem., 17:191-201 (1993)) low-complexity filters can be employed alone or in combination.

(c) As used herein, “sequence identity” or “identity” in the context of two nucleic acid or polypeptide sequences includes reference to the residues in the two sequences which are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are said to have “sequence similarity” or “similarity”. Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, Computer Applic. Biol. Sci., 4:11-17 (1988) e.g., as implemented in the program PC/GENE (Intelligenetics, Mountain View, Calif., USA).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

(e)(I) The term “substantial identity” of polynucleotide sequences means that a polynucleotide comprises a sequence that has at least 70% sequence identity, preferably at least 80%, more preferably at least 90% and most preferably at least 95%, compared to a reference sequence using one of the alignment programs described using standard parameters. One of skill will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of at least 60%, or preferably at least 70%, 80%, 90%, and most preferably at least 95%.

These programs and algorithms can ascertain the analogy of a particular polymorphism in a target gene to those disclosed herein. It is expected that this polymorphism will exist in other animals and use of the same in other animals than disclosed herein involved no more than routine optimization of parameters using the teachings herein.

It is also possible to establish linkage between specific alleles of alternative DNA markers and alleles of DNA markers known to be associated with a particular gene (e.g. the genes discussed herein), which have previously been shown to be associated with a particular trait. Thus, in the present situation, taking one or both of the genes, it would be possible, at least in the short term, to select for animals likely to produce desired traits, or alternatively against animals likely to produce less desirable traits indirectly, by selecting for certain alleles of an associated marker through the selection of specific alleles of alternative chromosome markers. As used herein the term “genetic marker” shall include not only the nucleotide polymorphisms disclosed by any means of assaying for the protein changes associated with the polymorphism, be they linked markers, use of microsatellites, or even other means of assaying for the causative protein changes indicated by the marker and the use of the same to influence traits of an animal.

As used herein, often the designation of a particular polymorphism is made by the name of a particular restriction enzyme. This is not intended to imply that the only way that the site can be identified is by the use of that restriction enzyme. There are numerous databases and resources available to those of skill in the art to identify other restriction enzymes which can be used to identify a particular polymorphism, for example http:Hldarwin.bio.geneseo.edu which can give restriction enzymes upon analysis of a sequence and the polymorphism to be identified. In fact as disclosed in the teachings herein there are numerous ways of identifying a particular polymorphism or allele with alternate methods which may not even include a restriction enzyme, but which assay for the same genetic or proteomic alternative form.

The accompanying Figures, which are incorporated herein and which constitute a part of this specification, illustrates one embodiment of the invention and, together with the description, serve to explain the principles of the invention.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

FIG. 1 shows the cDNA sequence that was isolated from a pig liver cDNA library and the predicted amino acid sequence. SULT1A1 cDNA was isolated from a pig liver cDNA library. The nucleotide sequence has been registered in GenBank (accession number, AY193893). The predicted amino acid sequence is indicated below the corresponding nucleotide sequence. The numbers of nucleotides and amino acids are indicated at the right. Polyadenylation signal (AATAAA) is underlined.

FIG. 2 shows an amino acid sequence comparison between pig phenol sulfotransferase and human SULT1A1, SULT1A2 and SULT1A3. Glu83, Asp134 and Asp263 are reported to be active sites for human SULT1A1. Gln121, Thr185, and Thr267 are common residues in phenol sulfotransferase. The asterisk indicates residues for the active sites between human and pig. The common residues of phenol sulfotransferase between human and pig are in bold.

FIG. 3 shows the sequence of the genetic polymorphism (B) and in vivo microsomal sulfation activity, and skatole level in fat (A). Liver micosomal sulfation activity and skatole level in fat for both substitution and wild type samples.

FIG. 4 shows sulfation activity of recombinant expressed proteins encoded by pig phenol sulfotransferase cDNA.

### DETAILED DESCRIPTION OF THE INVENTION

Reference will now be made in detail to the presently referred embodiments of the invention, which together with the following examples, serve to explain the principles of the invention.

The invention relates to genetic markers and methods of identifying those markers in an animal of a particular breed, strain, population, or group, whereby the animal is more likely to yield desired boar taint traits.

According to the invention, the genes encoding sulfotransferase enzymes which are involved in skatole metabolism have been identified as major effect genes. Variation in these genes has a measurable effect on boar taint in pigs. Thus screening methods may be developed for variation within or linked to these genes that are predictive of phenotypic variation.

In pigs, it has been found that a plasma concentration of 6-sulfatoxyskatole, the sulfoconjugate of 6-hydroxyskatole produced by phase II metabolism by sulfotransferase, is positively correlated to clearing skatole. (Babol et al., 1998). The capability of synthesis of 6-sulfatoxyskatole is a major step in a rapid metabolic clearance of skatole, resulting in low concentrations of skatole in fat and further low level of boar taint. Therefore, sulfotransferase plays an important role in the metabolism and clearance of skatole from the body in pigs.

Sulfation is one of the major conjugation reactions involved in the metabolism of many hormones, neurotransmitters, drugs, and xenobiotic compounds (Winshilboum et al., 1997; Her et al, 1996; Dooley, 1998). Phenol sulfortransferase is considered to be the most important enzyme that catalyzes sulfate conjugation (Dooley, 1998). In humans, phenol sulfotransferase is expressed in many tissues including liver, spleen, lung, testis, kidney, skin, brain, adrenal gland, olfactory epithelium, and platelets. The expression of this gene in many tissues shows its importance in life process in vivo.

The molecular biology of phenol sulfotransferase has advanced rapidly. The phenol sulfotransferase genes in human (Her et al 1996), mouse (Sakakibara et al, 1998), rat (access number: AF394783) and bovine (Henry et al., 1996) have been isolated and characterized.

Functionally significant genetic polymorphisms for phenol sulfotransferase enzymes have been reported in humans, and other molecular genetic mechanisms that might be involved in the regulation of the expression of these enzymes have been explored (Chen et al, 2000; Seth, et al, 2000; Dooley, 1998). In humans, knowledge of the molecular biology of phenol sulfotransferase enzymes promises to significantly improve the understanding of the regulation of the sulfate conjugation of hormones, neurotransmitters, drugs, and xenobiotic compounds, in order to diagnose lung cancer, protect against colorectal cancers and breast cancers (Wang et al. 2002; Bamber et al, 2001; Seth et al, 2000). In pigs, it has been reported that phenol sulfotransferase is negatively correlated with skatole accumulation in fat (Babol et al, 1998, Diaz and squires, 2003). Pigs with high sulfation activity have low level of skatole in fat, vice verse. Thus changes in the activity of the sulfation metabolic pathway could be used as genetic marker to select for skatole metabolism in pigs. However, the information about phenol sulfotransferase gene, its expression and how a genetic variation in this enzyme translates into interindividual variation in skatole level in pigs is unknown.

According to the invention a cDNA library was constructed from pig liver by rapid amplification of cDNA ends (RACE) and the sequence of porcine SULT1A1 cDNA was determined. The expression pattern of the SUTL1A1 mRNA species was examined in different tissues in pigs by RT-PCR. The polymerase chain reaction technique combined with single strand conformational polymorphism (PCR-SSCP) was used to scan for polymorphisms in the SULT1A1 coding region from porcine liver tissues, which may alter the metabolic capacities of the enzyme. We have identified a substitution mutation A→G in the coding region of the SULT1A1 gene that codes for a Lys¹⁴⁷ Glu¹⁴⁷. Functional characterization of this mutant was carried out by transfection into a COS-7 cell line.

According to the invention, the association of alternate forms of sulfotransferase enzymes may be used to identify and select pigs with differences in boar taint. For example, according to the invention, an allele of the sulfotransferase gene has been identified that results in a protein change and increase activity of the sulfotransferase enzyme, which leads to lower skatole levels in the pig.

Further according to the invention, other polymorphisms sulfotransferase genes in the pig may be identified to genetically type and select pigs based upon their proclivity to boar taint. Many factors can influence a metabolic pathway, some products are the result of rate limiting substrates or enzymes and it is unpredictable which enzymes may have variability that will result in an actual increase of a reaction product and thus a phenotypic trait. Once an association between a particular gene or gene product in the pathway and protein activity that affects the resultant trait is made, genes encoding these proteins may be screened for other polymorphisms or markers which may be used to indicate differences in these animals with respect to the trait. The active sites of these enzymes are the most susceptible to variability that will cause a significant affect in the metabolic products. These polymorphisms with these genes enable genetic markers to be identified for specific breeds or genetic lines or animals, boar taint potential early in the animal's life.

An alternate form of sulfotransferase has been identified according to the invention which results in an amino acid change and decreased enzyme activity causing higher skatole levels in the pig. Tests for the presence of this alternate form may be developed using the novel sequence for sulfotransferase as disclosed herein. These tests include but are not limited to PCR, SSCP, and the like.

Thus, the invention relates to genetic markers and methods of identifying those markers in an animal of a particular animal, breed, strain, population, or group, whereby the animal is has increased, decreased or otherwise altered skatole metabolism, and thus boar taint.

Any method of identifying the presence or absence of these markers may be used, including, for example, single-strand conformation polymorphism (SSCP) analysis, base excision sequence scanning (BESS), RFLP analysis, heteroduplex analysis, denaturing gradient gel electrophoresis, and temperature gradient electrophoresis, allelic PCR, ligase chain reaction direct sequencing, mini sequencing, nucleic acid hybridization, micro-array-type detection of genes encoding enzymes involved in skatole metabolism. Also within the scope of the invention includes assaying for protein conformational or sequences changes which occur in the presence of this polymorphism. The polymorphism may or may not be the causative mutation but will be indicative of the presence of this change and one may assay for the genetic or protein bases for the phenotypic difference.

The following is a general overview of techniques which can be used to assay for the genetic marker of the invention.

In the present invention, a sample of genetic material is obtained from an animal. Samples can be obtained from blood, tissue, semen, etc. Generally, peripheral blood cells are used as the source, and the genetic material is DNA. A sufficient amount of cells are obtained to provide a sufficient amount of DNA for analysis. This amount will be known or readily determinable by those skilled in the art. The DNA is isolated from the blood cells by techniques known to those skilled in the art.

Isolation and Amplification of Nucleic Acid

Samples of genomic DNA are isolated from any convenient source including saliva, buccal cells, hair roots, blood, cord blood, amniotic fluid, interstitial fluid, peritoneal fluid, chorionic villus, and any other suitable cell or tissue sample with intact interphase nuclei or metaphase cells. The cells can be obtained from solid tissue as from a fresh or preserved organ or from a tissue sample or biopsy. The sample can contain compounds which are not naturally intermixed with the biological material such as preservatives, anticoagulants, buffers, fixatives, nutrients, antibiotics, or the like.

Methods for isolation of genomic DNA from these various sources are described in, for example, Kirby, DNA Fingerprinting, An Introduction, W.H. Freeman & Co. New York (1992). Genomic DNA can also be isolated from cultured primary or secondary cell cultures or from transformed cell lines derived from any of the aforementioned tissue samples.

Samples of animal RNA can also be used. RNA can be isolated from tissues expressing the gene as described in Sambrook et al., supra. RNA can be total cellular RNA, mRNA, poly A+ RNA, or any combination thereof. For best results, the RNA is purified, but can also be unpurified cytoplasmic RNA. RNA can be reverse transcribed to form DNA which is then used as the amplification template, such that the PCR indirectly amplifies a specific population of RNA transcripts. See, e.g., Sambrook, supra, Kawasaki et al., Chapter 8 in PCR Technology, (1992) supra, and Berg et al., Hum. Genet. 85:655-658 (1990).

PCR Amplification

The most common means for amplification is polymerase chain reaction (PCR), as described in U.S. Pat. Nos. 4,683,195; 4,683,202; and 4,965,188 each of which is hereby incorporated by reference. If PCR is used to amplify the target regions in blood cells, heparinized whole blood should be drawn in a sealed vacuum tube kept separated from other samples and handled with clean gloves. For best results, blood should be processed immediately after collection; if this is impossible, it should be kept in a sealed container at 4° C. until use. Cells in other physiological fluids may also be assayed. When using any of these fluids, the cells in the fluid should be separated from the fluid component by centrifugation.

Tissues should be roughly minced using a sterile, disposable scalpel and a sterile needle (or two scalpels) in a 5 mm Petri dish. Procedures for removing paraffin from tissue sections are described in a variety of specialized handbooks well known to those skilled in the art.

To amplify a target nucleic acid sequence in a sample by PCR, the sequence must be accessible to the components of the amplification system. One method of isolating target DNA is crude extraction which is useful for relatively large samples. Briefly, mononuclear cells from samples of blood, amniocytes from amniotic fluid, cultured chorionic villus cells, or the like are isolated by layering on a sterile Ficoll-Hypaque gradient by standard procedures. Interphase cells are collected and washed three times in sterile phosphate buffered saline before DNA extraction. If testing DNA from peripheral blood lymphocytes, an osmotic shock (treatment of the pellet for 10 sec with distilled water) is suggested, followed by two additional washings if residual red blood cells are visible following the initial washes. This will prevent the inhibitory effect of the heme group carried by hemoglobin on the PCR reaction. If PCR testing is not performed immediately after sample collection, aliquots of 10⁶ cells can be pelleted in sterile Eppendorf tubes and the dry pellet frozen at −20° C. until use.

The cells are resuspended (10⁶ nucleated cells per 100 μl) in a buffer of 50 mM Tris-HCl (pH 8.3), 50 mM KCl 1.5 mM MgCl₂, 0.5% Tween 20, and 0.5% NP40 supplemented with 100 μg/ml of proteinase K. After incubating at 56° C. for 2 hr. the cells are heated to 95° C. for 10 min to inactivate the proteinase K and immediately moved to wet ice (snap-cool). If gross aggregates are present, another cycle of digestion in the same buffer should be undertaken. Ten μl of this extract is used for amplification.

When extracting DNA from tissues, e.g., chorionic villus cells or confluent cultured cells, the amount of the above mentioned buffer with proteinase K may vary according to the size of the tissue sample. The extract is incubated for 4-10 hrs at 50°−60° C. and then at 95° C. for 10 minutes to inactivate the proteinase. During longer incubations, fresh proteinase K should be added after about 4 hr at the original concentration.

When the sample contains a small number of cells, extraction may be accomplished by methods as described in Higuchi, “Simple and Rapid Preparation of Samples for PCR”, in PCR Technology, Ehrlich, H. A. (ed.), Stockton Press, New York, which is incorporated herein by reference. PCR can be employed to amplify target regions in very small numbers of cells (1000-5000) derived from individual colonies from bone marrow and peripheral blood cultures. The cells in the sample are suspended in 20 μl of PCR lysis buffer (10 mM Tris-HCl (pH 8.3), 50 mM KCl, 2.5 mM MgCl₂, 0.1 mg/ml gelatin, 0.45% NP40, 0.45% Tween 20) and frozen until use. When PCR is to be performed, 0.6 μl of proteinase K (2 mg/ml) is added to the cells in the PCR lysis buffer. The sample is then heated to about 60° C. and incubated for 1 hr. Digestion is stopped through inactivation of the proteinase K by heating the samples to 95° C. for 10 min and then cooling on ice.

A relatively easy procedure for extracting DNA for PCR is a salting out procedure adapted from the method described by Miller et al., Nucleic Acids Res. 16:1215 (1988), which is incorporated herein by reference. Mononuclear cells are separated on a Ficoll-Hypaque gradient. The cells are resuspended in 3 ml of lysis buffer (10 mM Tris-HCl, 400 mM NaCl, 2 mM Na₂ EDTA, pH 8.2). Fifty μl of a 20 mg/ml solution of proteinase K and 150 μl of a 20% SDS solution are added to the cells and then incubated at 37° C. overnight. Rocking the tubes during incubation will improve the digestion of the sample. If the proteinase K digestion is incomplete after overnight incubation (fragments are still visible), an additional 50 μl of the 20 mg/ml proteinase K solution is mixed in the solution and incubated for another night at 37° C. on a gently rocking or rotating platform. Following adequate digestion, one ml of a 6M NaCl solution is added to the sample and vigorously mixed. The resulting solution is centrifuged for 15 minutes at 3000 rpm. The pellet contains the precipitated cellular proteins, while the supernatant contains the DNA. The supernatant is removed to a 15 ml tube that contains 4 ml of isopropanol. The contents of the tube are mixed gently until the water and the alcohol phases have mixed and a white DNA precipitate has formed. The DNA precipitate is removed and dipped in a solution of 70% ethanol and gently mixed. The DNA precipitate is removed from the ethanol and air-dried. The precipitate is placed in distilled water and dissolved.

Kits for the extraction of high-molecular weight DNA for PCR include a Genomic Isolation Kit A.S.A.P. (Boehringer Mannheim, Indianapolis, Ind.), Genomic DNA Isolation System (GIBCO BRL, Gaithersburg, Md.), Elu-Quik DNA Purification Kit (Schleicher & Schuell, Keene, N.H.), DNA Extraction Kit (Stratagene, LaJolla, Calif.), TurboGen Isolation Kit (Invitrogen, San Diego, Calif.), and the like. Use of these kits according to the manufacturer's instructions is generally acceptable for purification of DNA prior to practicing the methods of the present invention.

The concentration and purity of the extracted DNA can be determined by spectrophotometric analysis of the absorbance of a diluted aliquot at 260 nm and 280 nm. After extraction of the DNA, PCR amplification may proceed. The first step of each cycle of the PCR involves the separation of the nucleic acid duplex formed by the primer extension. Once the strands are separated, the next step in PCR involves hybridizing the separated strands with primers that flank the target sequence. The primers are then extended to form complementary copies of the target strands. For successful PCR amplification, the primers are designed so that the position at which each primer hybridizes along a duplex sequence is such that an extension product synthesized from one primer, when separated from the template (complement), serves as a template for the extension of the other primer. The cycle of denaturation, hybridization, and extension is repeated as many times as necessary to obtain the desired amount of amplified nucleic acid.

In a particularly useful embodiment of PCR amplification, strand separation is achieved by heating the reaction to a sufficiently high temperature for a sufficient time to cause the denaturation of the duplex but not to cause an irreversible denaturation of the polymerase (see U.S. Pat. No. 4,965,188, incorporated herein by reference). Typical heat denaturation involves temperatures ranging from about 80° C. to 105° C. for times ranging from seconds to minutes. Strand separation, however, can be accomplished by any suitable denaturing method including physical, chemical, or enzymatic means. Strand separation may be induced by a helicase, for example, or an enzyme capable of exhibiting helicase activity. For example, the enzyme RecA has helicase activity in the presence of ATP. The reaction conditions suitable for strand separation by helicases are known in the art (see Kuhn Hoffman-Berling, 1978, CSH-Quantitative Biology, 43:63-67; and Radding, 1982, Ann. Rev. Genetics 16:405-436, each of which is incorporated herein by reference).

Template-dependent extension of primers in PCR is catalyzed by a polymerizing agent in the presence of adequate amounts of four deoxyribonucleotide triphosphates (typically dATP, dGTP, dCTP, and dTTP) in a reaction medium comprised of the appropriate salts, metal cations, and pH buffering systems. Suitable polymerizing agents are enzymes known to catalyze template-dependent DNA synthesis. In some cases, the target regions may encode at least a portion of a protein expressed by the cell. In this instance, mRNA may be used for amplification of the target region. Alternatively, PCR can be used to generate a cDNA library from RNA for further amplification, the initial template for primer extension is RNA. Polymerizing agents suitable for synthesizing a complementary, copy-DNA (cDNA) sequence from the RNA template are reverse transcriptase (RT), such as avian myeloblastosis virus RT, Moloney murine leukemia virus RT, or Thermus thermophilus (Tth) DNA -polymerase, a thermostable DNA polymerase with reverse transcriptase activity marketed by Perkin Elmer Cetus, Inc. Typically, the genomic RNA template is heat degraded during the first denaturation step after the initial reverse transcription step leaving only DNA template. Suitable polymerases for use with a DNA template include, for example, E. coli DNA polymerase I or its Klenow fragment, T4 DNA polymerase, Tth polymerase, and Taq polymerase, a heat-stable DNA polymerase isolated from Thermus aquaticus and commercially available from Perkin Elmer Cetus, Inc. The latter enzyme is widely used in the amplification and sequencing of nucleic acids. The reaction conditions for using Taq polymerase are known in the art and are described in Gelfand, 1989, PCR Technology, supra.

Allele Specific PCR

Allele-specific PCR differentiates between target regions differing in the presence of absence of a variation or polymorphism. PCR amplification primers are chosen which bind only to certain alleles of the target sequence. This method is described by Gibbs, Nucleic Acid Res. 17:12427-2448 (1989).

Allele Specific Oligonucleotide Screening Methods

Further diagnostic screening methods employ the allele-specific oligonucleotide (ASO) screening methods, as described by Saiki et al., Nature 324:163-166 (1986). Oligonucleotides with one or more base pair mismatches are generated for any particular allele. ASO screening methods detect mismatches between variant target genomic or PCR amplified DNA and non-mutant oligonucleotides, showing decreased binding of the oligonucleotide relative to a mutant oligonucleotide. Oligonucleotide probes can be designed so that under low stringency, they will bind to both polymorphic forms of the allele, but at high stringency, bind to the allele to which they correspond. Alternatively, stringency conditions can be devised in which an essentially binary response is obtained, i.e., an ASO corresponding to a variant form of the target gene will hybridize to that allele, and not to the wild-type allele.

Ligase Mediated Allele Detection Method

Target regions of a test subject's DNA can be compared with target regions in unaffected and affected family members by ligase-mediated allele detection. See Landegren et al., Science 241:107-1080 (1988). Ligase may also be used to detect point mutations in the ligation amplification reaction described in Wu et al., Genomics 4:560-569 (1989). The ligation amplification reaction (LAR) utilizes amplification of specific DNA sequence using sequential rounds of template dependent ligation as described in Wu, supra, and Barany, Proc. Nat. Acad. Sci. 88:189-193 (1990).

Denaturing Gradient Gel Electrophoresis

Amplification products generated using the polymerase chain reaction can be analyzed by the use of denaturing gradient gel electrophoresis. Different alleles can be identified based on the different sequence-dependent melting properties and electrophoretic migration of DNA in solution. DNA molecules melt in segments, termed melting domains, under conditions of increased temperature or denaturation. Each melting domain melts cooperatively at a distinct, base-specific melting temperature (Tₘ). Melting domains are at least 20 base pairs in length, and may be up to several hundred base pairs in length.

Differentiation between alleles based on sequence specific melting domain differences can be assessed using polyacrylamide gel electrophoresis, as described in Chapter 7 of Erlich, ed., PCR Technology, “Principles and Applications for DNA Amplification”. W.H. Freeman and Co., New York (1992), the contents of which are hereby incorporated by reference.

Generally, a target region to be analyzed by denaturing gradient gel electrophoresis is amplified using PCR primers flanking the target region. The amplified PCR product is applied to a polyacrylamide gel with a linear denaturing gradient as described in Myers et al., Meth. Enzymol. 155:501-527 (1986), and Myers et al., in Genomic Analysis, A Practical Approach, K. Davies Ed. IRL Press Limited, Oxford, pp. 95-139 (1988), the contents of which are hereby incorporated by reference. The electrophoresis system is maintained at a temperature slightly below the Tm of the melting domains of the target sequences.

In an alternative method of denaturing gradient gel electrophoresis, the target sequences may be initially attached to a stretch of GC nucleotides, termed a GC clamp, as described in Chapter 7 of Erlich, supra. Preferably, at least 80% of the nucleotides in the GC clamp are either guanine or cytosine. Preferably, the GC clamp is at least 30 bases long. This method is particularly suited to target sequences with high Tₘ′s.

Generally, the target region is amplified by the polymerase chain reaction as described above. One of the oligonucleotide PCR primers carries at its 5′ end, the GC clamp region, at least 30 bases of the GC rich sequence, which is incorporated into the 5′ end of the target region during amplification. The resulting amplified target region is run on an electrophoresis gel under denaturing gradient conditions as described above. DNA fragments differing by a single base change will migrate through the gel to different positions, which may be visualized by ethidium bromide staining.

Temperature Gradient Gel Electrophoresis

Temperature gradient gel electrophoresis (TGGE) is based on the same underlying principles as denaturing gradient gel electrophoresis, except the denaturing gradient is produced by differences in temperature instead of differences in the concentration of a chemical denaturant. Standard TGGE utilizes an electrophoresis apparatus with a temperature gradient running along the electrophoresis path. As samples migrate through a gel with a uniform concentration of a chemical denaturant, they encounter increasing temperatures. An alternative method of TGGE, temporal temperature gradient gel electrophoresis (TTGE or tTGGE) uses a steadily increasing temperature of the entire electrophoresis gel to achieve the same result. As the samples migrate through the gel the temperature of the entire gel increases, leading the samples to encounter increasing temperature as they migrate through the gel. Preparation of samples, including PCR amplification with incorporation of a GC clamp, and visualization of products are the same as for denaturing gradient gel electrophoresis.

Single-Strand Conformation Polymorphism Analysis

Target sequences or alleles at the chosen boar taint loci can be differentiated using single-strand conformation polymorphism analysis, which identifies base differences by alteration in electrophoretic migration of single-stranded PCR products, as described in Orita et al., Proc. Nat. Acad. Sci. 85:2766-2770 (1989). Amplified PCR products can be generated as described above, and heated or otherwise denatured, to form single-stranded amplification products. Single-stranded nucleic acids may refold or form secondary structures which are partially dependent on the base sequence. Thus, electrophoretic mobility of single-stranded amplification products can detect base-sequence difference between alleles or target sequences.

Chemical or Enzymatic Cleavage of Mismatches

Differences between target sequences can also be detected by differential chemical cleavage of mismatched base pairs, as described in Grompe et al., Am. J. Hum. Genet. 48:212-222 (1991). In another method, differences between target sequences can be detected by enzymatic cleavage of mismatched base pairs, as described in Nelson et al., Nature Genetics 4:11-18 (1993). Briefly, genetic material from an animal and an affected family member may be used to generate mismatch free heterohybrid DNA duplexes. As used herein, “heterohybrid” means a DNA duplex strand comprising one strand of DNA from one animal, and a second DNA strand from another animal, usually an animal differing in the phenotype for the trait of interest. Positive selection for heterohybrids free of mismatches allows determination of small insertions, deletions or other polymorphisms that may be associated with polymorphisms.

Non-Gel Systems

Other possible techniques include non-gel systems such as TAQMAN™ (Perkin Elmer). In this system, oligonucleotide PCR primers are designed that flank the mutation in question and allow PCR amplification of the region. A third oligonucleotide probe is then designed to hybridize to the region containing the base subject to change between different alleles of the gene. This probe is labeled with fluorescent dyes at both the 5′ and 3′ ends. These dyes are chosen such that while in this proximity to each other the fluorescence of one of them is quenched by the other and cannot be detected. Extension by Taq DNA polymerase from the PCR primer positioned 5′ on the template relative to the probe leads to the cleavage of the dye attached to the 5′ end of the annealed probe through the 5′ nuclease activity of the Taq DNA polymerase. This removes the quenching effect allowing detection of the fluorescence from the dye at the 3′ end of the probe. The discrimination between different DNA sequences arises through the fact that if the hybridization of the probe to the template molecule is not complete, i.e., there is a mismatch of some form, the cleavage of the dye does not take place. Thus, only if the nucleotide sequence of the oligonucleotide probe is completely complimentary to the template molecule to which it is bound will quenching be removed. A reaction mix can contain two different probe sequences each designed against different alleles that might be present thus allowing the detection of both alleles in one reaction.

Yet another technique includes an Invader Assay, which includes isothermic amplification that relies on a catalytic release of fluorescence. See Third Wave Technology at www.twt.com.

Non-PCR Based DNA Diagnostics

The identification of a DNA sequence linked to sequences encoding enzymes involved in skatole metabolism can be made without an amplification step, based on polymorphisms including restriction fragment length polymorphisms in an animal and a family member. Hybridization probes are generally oligonucleotides which bind through complementary base pairing to all or part of a target nucleic acid. Probes typically bind target sequences lacking complete complementarity with the probe sequence depending on the stringency of the hybridization conditions. The probes are preferably labeled directly or indirectly, such that by assaying for the presence or absence of the probe, one can detect the presence or absence of the target sequence. Direct labeling methods include radioisotope labeling, such as with p³² or S³⁵. Indirect labeling methods include fluorescent tags, biotin complexes which may be bound to avidin or streptavidin, or peptide or protein tags. Visual detection methods include photoluminescents, Texas red, rhodamine and its derivatives, red leuco dye and 3,3′,5,5′-tetramethylbenzidine (TMB), fluorescein, and its derivatives, dansyl, umbelliferone and the like or with horse radish peroxidase, alkaline phosphatase and the like.

Hybridization probes include any nucleotide sequence capable of hybridizing to the porcine chromosome where the sulfotransferase gene or other gene involved in skatole metabolism resides, and thus defining a genetic marker linked to the gene, including a restriction fragment length polymorphism, a hypervariable region, repetitive element, or a variable number tandem repeat. Hybridization probes can be any gene or a suitable analog. Further suitable hybridization probes include exon fragments or portions of cDNAs or genes known to map to the relevant region of the chromosome.

Preferred tandem repeat hybridization probes for use according to the present invention are those that recognize a small number of fragments at a specific locus at high stringency hybridization conditions, or that recognize a larger number of fragments at that locus when the stringency conditions are lowered.

One or more additional restriction enzymes and/or probes and/or primers can be used. Additional enzymes, constructed probes, and primers can be determined by routine experimentation by those of ordinary skill in the art and are intended to be within the scope of the invention.

According to the invention, polymorphisms in genes encoding enzymes involved in skatole metabolism have been identified which have an association with boar taint. The presence or absence of the markers, in one embodiment may be assayed by PCR-RFLP analysis using the restriction endonucleases and amplification primers may be designed using analogous human, pig or other sequences due to the high homology in the region surrounding the polymorphisms, or may be designed using known gene sequence data as exemplified in GenBank or even designed from sequences obtained from linkage data from closely surrounding genes based upon the teachings and references herein. The sequences surrounding the polymorphism will facilitate the development of alternate PCR tests in which a primer of about 4-30 contiguous bases taken from the sequence immediately adjacent to the polymorphism is used in connection with a polymerase chain reaction to greatly amplify the region before treatment with the desired restriction enzyme. The primers need not be the exact complement; substantially equivalent sequences are acceptable. The design of primers for amplification by PCR is known to those of skill in the art and is discussed in detail in Ausubel (ed.), Short Protocols in Molecular Biology, 4th Edition, John Wiley and Sons (1999).

The following is a brief description of primer design. Generally the primers used for the assays of the invention will flank nt 546 on each side, one forward and one reverse.

Primer Design Strategy

Increased use of polymerase chain reaction (PCR) methods has stimulated the development of many programs to aid in the design or selection of oligonucleotides used as primers for PCR. Four examples of such programs that are freely available via the Internet are: PRIMER by Mark Daly and Steve Lincoln of the Whitehead Institute (UNIX, VMS, DOS, and Macintosh), Oligonucleotide Selection Program (OSP) by Phil Green and LaDeana Hiller of Washington University in St. Louis (UNIX, VMS, DOS, and Macintosh), PGEN by Yoshi (DOS only), and Amplify by Bill Engels of the University of Wisconsin (Macintosh only). Generally these programs help in the design of PCR primers by searching for bits of known repeated-sequence elements and then optimizing the Tₘ by analyzing the length and GC content of a putative primer. Commercial software is also available and primer selection procedures are rapidly being included in most general sequence analysis packages.

Sequencing and PCR Primers

Designing oligonucleotides for use as either sequencing or PCR primers requires selection of an appropriate sequence that specifically recognizes the target, and then testing the sequence to eliminate the possibility that the oligonucleotide will have a stable secondary structure. Inverted repeats in the sequence can be identified using a repeat-identification or RNA-folding program such as those described above. If a possible stem structure is observed, the sequence of the primer can be shifted a few nucleotides in either direction to minimize the predicted secondary structure. The sequence of the oligonucleotide should also be compared with the sequences of both strands of the appropriate vector and insert DNA. Obviously, a sequencing primer should only have a single match to the target DNA. It is also advisable to exclude primers that have only a single mismatch with an undesired target DNA sequence. For PCR primers used to amplify genomic DNA, the primer sequence should be compared to the sequences in the GenBank database to determine if any significant matches occur. If the oligonucleotide sequence is present in any known DNA sequence or, more importantly, in any known repetitive elements, the primer sequence should be changed.

The methods and materials of the invention may also be used more generally to evaluate pig DNA, genetically type individual pigs, and detect genetic differences in pigs. In particular, a sample of pig genomic DNA may be evaluated by reference to one or more controls to determine if a polymorphism in the particular gene is present. Preferably, RFLP analysis is performed with respect to the pig gene, and the results are compared with a control. The control is the result of a RFLP analysis of the pig gene of a different pig where the polymorphism(s) of the pig gene is/are known. Similarly, the genotype of a pig may be determined by obtaining a sample of its genomic DNA, conducting RFLP analysis of the gene in the DNA, and comparing the results with a control. Again, the control is the result of RFLP analysis of the gene of a different pig. The results genetically type the pig by specifying the polymorphism(s) in its genes. Finally, genetic differences among pigs can be detected by obtaining samples of the genomic DNA from at least two pigs, identifying the presence or absence of a polymorphism in the gene, and comparing the results.

These assays are useful for identifying the genetic markers relating to boar taint, , as discussed above, for identifying other polymorphisms in the genes encoding enzymes involved in skatole metabolism and for the general scientific analysis of pig genotypes and phenotypes.

The examples and methods herein disclose certain gene(s) which has been identified to have a polymorphism(s) which is associated either positively or negatively with a beneficial trait that will have an effect on boar taint for animals carrying this polymorphism. The identification of the existence of a polymorphism within a gene is often made by a single base alternative that results in a restriction site in certain allelic forms. A certain allele, however, as demonstrated and discussed herein, may have a number of base changes associated with it that could be assayed for which are indicative of the same polymorphism (allele). Further, other genetic markers or genes may be linked to the polymorphisms disclosed herein so that assays may involve identification of other genes or gene fragments, but which ultimately rely upon genetic characterization of animals for the same polymorphism. Any assay which sorts and identifies animals based upon the allelic differences disclosed herein are intended to be included within the scope of this invention.

One of skill in the art, once a polymorphism has been identified and a correlation to a particular trait established will understand that there are many ways to genotype animals for this polymorphism. The design of such alternative tests merely represents optimization of parameters known to those of skill in the art and is intended to be within the scope of this invention as fully described herein.

The following non-limiting examples are illustrative of the present invention:

### EXAMPLES

Tissue Samples

A liver tissue was obtained from a male pig for construction of cDNA library. To identify genetic polymorphisms in SULT1A1 gene, liver tissues were obtained from sixty-nine intact male pigs from a variety of breeds, including Yorkshire, Duroc, Landrace, and Pietrain, as well as crosses between Landrace and Duroc, Large White and Duroc, and Large White and Pertain. The animals were slaughtered at an average live weight of 144±33 kg. A sample of liver was taken immediately following exsanguination, frozen in liquid nitrogen and stored at −70° C. before use. For measuring the expression profile of SULT1A1 mRNA, tissues including spleen, thymus, liver, lung, muscle, kidney, small intestine, heart, ovaries and testis were collected from one Landrace boar and one Landrace female that weighed approximately 100 kg.

Measurement of Skatole Level in Fat

A backfat sample was collected at the midline point of 11th rib and frozen at −20° C. until assayed for skatole. The skatole content was measured with a HPLC assay, according to the method described by Diaz and Squires (2000).

Isolation of Total RNA

One hundred milligrams of each tissue sample was homogenized in 1 ml of Tri-Reagent (Sigma, ST. Louis, Mo.) and incubated for 10 minutes at room temperature. After incubation, 0.2 ml of chloroform was added and the samples were vortexed and then centrifuged at 12,000×g for 10 minutes at 4° C. The aqueous phase was transferred into a sterile tube and mixed with 0.5 ml of isopropanol and incubated at room temperature for 10 minutes. The samples were centrifuged at 12,000×g for 10 minutes at 4° C. to precipitate the RNA. The pellet was washed with 75% ethanol and then suspended into 50 μl of DEPC water.

Construction and Screening of a Pig cDNA RACE Library

5′ and 3′ rapid amplification of cDNAs (RACE) were constructed from 1 μg of total RNA from liver with the use of Smart RACE cDNA Amplification kit (BD Biosciences, Palo Alto, Calif.), and used as templates in the subsequent PCR screening of porcine phenol sulfotransferase cDNA. The 5′ RACE was performed by synthesizing the first strand cDNA with a modified lock-docking oligo (dT) primer and then tailing the product 5′ AAG CAG TGG TAT CAA CGC AGA GTA CGC GGG 3′ (anchor primer) in the 5′ end via terminal transferase. The 3′ RACE was performed with oligo (dT) primer but including the same lock-docking nucleotide positions as in the 5′ RACE. The cDNA fragments of porcine phenol sulfotransferase were amplified with anchor primer and the primers (A and B) designed from human SULT1A1 and SULT1A2 cDNA sequences. Primer A was 5′ CAC AGC TCA GAG CGG AAG C 3′ and primer B was 5′ AGT GGT GGG AGC TGC GTC ACA C 3′. To obtain the full-length porcine phenol sulfotransferase cDNA, the following primers were used in the subsequent PCR-based screening: primer A and anchor primer with 5′ Race as a template (annealing 61° C.); primer B and anchor primer with 3′ Race as a template (annealing 63° C.). The PCR consisted of 30 cycles of denaturing for 1 minute at 94° C., optimal annealing for 1 minute, and extending for 1 minute, with a final 10 minute extension step at 72° C. Ten microliters of the PCR products were analyzed by electrophoresis on a 1% agarose gel.

Colony Hybridization

When multiple bands were amplified from both 3′ and 5′ Race templates, the PCR products were cloned into pGEM-T Easy Vector System (Promega, Madison, Wis.), and subjected to colony hybridization to confirm the specificity of amplified fragment prior to DNA sequencing. Colonies were lifted from the positively charged nylon membrane (Roche, Indianapolis, Ind.)), and subjected to lysis and fixation in 0.5M NaCl for 5 minutes, followed by rinsing in 5×SSC for 1 minute, and allowed to air dried. Colony hybridization was performed with the ECL nucleotide DNA labeling and detection kit (Amersham Biosciences, Piscataway, N.J.). The probe used in the hybridization was the fragment amplified by primer A and primer B designed from the human SULT1A1 and SULT1A2 cDNAs. Thermal cycling consisted of: (1) 5 cycles of 94° C. for 30 sec and 72° C. for 3 min; (2) 5 cycles of 94° C. for 30 sec, 70° C. for 30 sec, and 72° C. for 3 min; (3) 25 cycles of 94° C. for 30 sec, 61° C. for 30 sec, and 72° C. for 3 min, with a final 72° C. extension for 10 min. After hybridization overnight at 42° C., the membrane was washed twice with 0.15×SSC for 20 minutes and exposed to x-ray film. The colony that gave the strongest signal was selected for sequencing.

Isolation of Full-Length Porcine Phenol Sulfotransferase cDNA

To obtain a full-length porcine phenol sulfotransferase sequence, the forward primer 5′ ATG GAG CCG GTC CAG GAC A 3′ and reverse primer 5′ TCA CAG CTC AGA GCG GAA GC 3′ were designed based on the sequence obtained from the 5′ and 3′ RACE. They were used to amplify the full-length porcine phenol sulfotransferase with either 5′ or 3′ RACE cDNA as a template. PCR profile was 3 min at 94° C., followed by 30 cycles of 1 min at 94° C., 1 min 30 sec at 63° C., 1 min at 72° C. and final extension at 72° C. The PCR fragment was cloned into T-Easy vector (Promega, Madison, Wis.) and subjected to sequence analysis.

Expression of Phenol Sulfotransferase Gene (SULT1A1) in Tissues

The tissue distribution of SULT1A1 mRNA was determined by RT-PCR. Total RNAs were isolated from 100 mg of porcine spleen, thymus, liver, lung, muscle, ovary, kidney, small intestine, heart, and testis tissues with Tri-Reagent (Sigma). Total RNAs were treated with DNase I (Ambion) for 20 minutes at 37° C. according to the product manual prior to RT-PCR. One microgram of treated total RNA from liver samples was used to synthesize the first strand cDNA by using SuperScript reverse transcriptase (Invitrogen) and oligo (dT) primer (Sigma). RT-PCR was carried out based on the method described below. The forward primer (5′ ATG GAG CCG GTC CAG GAC A 3′) and reverse primer (5′ TCA CAG CTC AGA GCG GAA GC 3′) were designed to amplify the entire coding region of porcine SULT1A1 gene. It corresponds to the product from the transcription start site (nucleotide position 108) to transcription stop site (nucleotide position 995), spanning 888 bp. Ten microliters of the PCR products were analyzed by electrophoresis on a 1% agarose gel.

Sequencing Analysis

The PCR fragments were ligated into pGEM-T Easy Vector System (Promega, Madison, Wis.), and then transformed into competent DH5α cells. DNAs were purified and subject to sequencing using an Applied Biosystems model ABI 377 DNA sequencer.

RT-PCR

To scan for genetic polymorphisms in the SULT1A1 gene, RT-PCR products that cover the whole coding region were amplified and then subjected to SSCP analysis. One to five micrograms of total RNA from liver samples were used to synthesize first strand cDNA using SuperScript reverse transcriptase (Invitrogen, Carlsbad, Calif.) and oligo (dT) primer (Sigma, ST. Louis, Mo.). Following the reverse transcription, 2.5 μl of the first strand cDNA was used as the template for PCR. The PCR mixtures (50 ul) contained 1×PCR buffer (100 mM Tris-HCl, pH 8.3; 500 mM KCl, 11 mM MgCl₂, 0.1% gelatin), 0.2 mM dNTP, 0.4 mM primers (forward and reverse primer) and 2.5 U of Red Taq polymerase (Sigma, ST. Louis, Mo.). The forward primer (5′ ATG GAG CCG GTC CAG GAC A 3′) and reverse primer (5′ TCA CAG CTC AGA GCG GAA GC 3′) were designed to amplify the entire coding region of SULT1A1 gene, which was based on our isolated SULT1A1 (GenBank accession number AY193893). The PCR profile was 3 minutes at 94° C., followed by 35 cycles of 1 minute at 94° C., 1 minute at 63° C., 1 minute at 72° C. and final extension of 10 minutes at 72° C.

Single-Strand Conformational Polymorphism (SSCP) Analysis

PCR products were first cut into fragments with KpnI enzyme, and then resolved by SSCP analysis. Ten microliters of amplified PCR product was digested with KpnI in a 25 μl reaction at 37° C. for 3 hours. A total of 7 μl of digested fragments were then diluted with 13 μl of loading buffer (10% sucrose, 0.01% bromophenol blue and 0.01% xylene cyanol FF). Each digestion reaction was denatured at 100° C. for 5 minutes, chilled on ice and resolved on a 10% polyacrylamide gel. The electrophoresis was carried out in a 130×160×1.0 mm vertical unit (Bio-Rad Laboratories, Hercules, Calif.), in 0.6×TBE buffer for 17 hours at 15° C. at 160 V. The gels were then silver stained.

Expression of the Phenol Sulfotransferase cDNA in COS-7 Cells

The expression vector, pcDNA3.1/V5-His TOPO TA Expression vector (Invitrogen), was used. The whole coding region of phenol sulfotransferase cDNA was amplified from the cDNA library with the following primers, forward: 5′ ATG GAG CCG GTC CAG GAC A 3′ (start codon bolded); reverse: 5′ TCA CAG CTC AGA GCG GAA GC 3′ (stop codon bolded). The PCR reaction was performed under the following conditions: 3 minutes at 94° C., followed by 30 cycles of 1 minute at 94° C, 1 minute at 63° C., 1 minute at 72° C., with a final 10 min extension step at 72° C. Following amplification, 50 μl of PCR product was purified by a QIAquick Nucleotide Removal kit (QIAGEN) and suspended in 30 μl of distilled water. Four microliters of purified PCR product was ligated to 1 μl (10 ng) of expression vector and incubated at room temperature for 30 minutes. The recombinant DNA was then transformed into TOP10 competent cells (Invitrogen), purified, and subjected to sequencing to confirm its orientation.

COS-7 cells, routinely maintained in Dulbecco's modified Eagle's medium (DMEM) containing 10% fetal bovine serum and 1% antibiotics, were used as the host cells for the expression of the recombinant protein. Dishes (150 mm) of COS-7 cells were individually transfected with 54 μg of recombinant DNA containing mutant (A→G at nucleotide 546 bp) and wild type porcine SULT1A1 cDNA using the Lipofectamine 2000 mediated procedure (Invitrogen), while COS-7 cells only and expression vector only were used as negative control. After transfection, the cells were incubated at 37° C., 5% CO2 for the first 18 hours without serum and antibiotics, and then incubated at 37° C., 5% CO2 in DMEM containing 10% fetal bovine serum, 1% antibiotics for 48 hours. At the end of incubation, the cells were rinsed twice with phosphate buffered saline and precipitated at 500 g for 5 minutes at 4° C. After discarding the supernatant, the precipitate was stored at −80° C. before assay for sulfotransferase activity.

Sulfotransdrase Activity Assay

ρ-nitrophenol was used as a substrate for the SULT1A1 enzymatic activity assay according to the method previously described (Diaz and Squires, 2003). The COS-7 cell pellets were lysed in buffer (50 mM Tris-HCl, 10 mM MgCl₂, 0.1 mM EDTA, pH 7.4) and sonicated for 20 sec. The protein concentrations were measured by Bio-Rad Protein assay. The reaction was run in a mixture of 4 mg/ml protein, 8 mM p-nitrophenol, 2 mM PAPS (Sigma) for 30 min at 37° C., terminated by adding an equal volume of ice-cold acetonitrile, vortexed and centrifuged to remove protein. One hundred microliters of supernatant was used to measure the formation of p-nitrophenyl sulfate by HPLC.

Sequence Characterization of Phenol Sulfotransferase (SULT1A1) cDNA

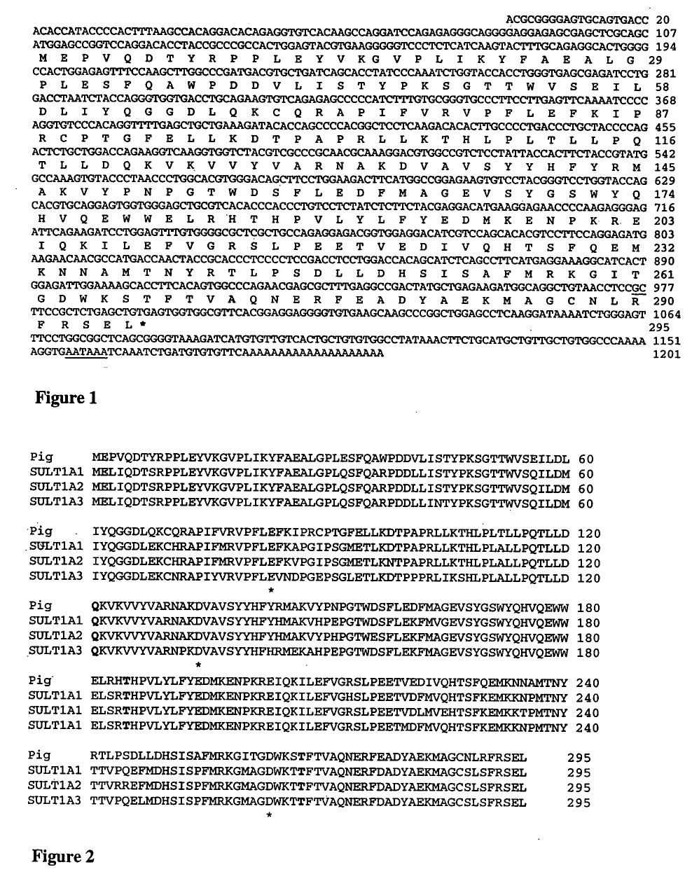

Porcine SULT1A1 cDNA was isolated by PCR screening of the liver cDNA library constructed with the RACE method. The nucleotide was 1201 bp long and contained an 888 bp-long open reading frame (ORF) encoding 296 amino acids and 206 bp long 3′ untranslated region including one polyadenylation signal, AATAAA (FIG. 1). Porcine SULT1A1 cDNA sequence was submitted to Genbank database under the accession number AY193893.

In humans, there are three highly homologous phenol sulfotransferases (PSTs) and three highly homologous (over 94%) PST genes, SULT1A1, SULT1A2, and SULT1A3 are located on chromosome 16p12.1. When we compared the pig phenol sulfotransferase coding region to the human genes, it showed 86% homology to SULT1A1 and SULT1A2, and 85% to SULT1A3. The deduced amino acid sequence for pig phenol sulfotransferase showed 86.7% homology to SULT1A1, 86.5% to SULT1A2, and 85.4% to SULT1A3 (FIG. 2). In humans, SULT1A1 , Glu83, Asp134 and Asp263 are reported to be the active site for SULT1A1 , and especially Glu83 and Asp134 are essential amino acids for SULT1A1 catalytic activity (Chen et al, 2000). Gln121, Thr185, and Thr267 are common residues in human phenol sulfotransferase (Honma et al, 2001). All the above active sites are conserved in the putative pig phenol sulfotransferase. To further characterize this gene, the recombinant protein encoded by this gene was expressed in COS-7 cells, and the enzyme activity of the expressed protein was assayed using ρ-nitrophenol as a substrate. These results indicate that this gene isolated from pig liver clearly represents phenol sulfotransferase.

Expression of Phenol Sulfotransferase mRNA in Various Tissues

The expression patterns of phenol sulfotransferase mRNA in spleen, thymus, liver, lung, muscle, ovary, kidney, small intestine, heart, and testis tissues of pigs were investigated by RT-PCR. To determine the mRNA level in tissues, the total RNA samples were treated with DNAse I to remove possible contamination with genomic DNA prior to RT-PCR. The result showed that phenol sulfotransferase (about 900 bp PCR products) was expressed in all of the 10 tissues examined except the small intestine (data not shown). This suggests that phenol sulfotransferase plays an important role in the life process in vivo in pigs.

Phenol Sulfotransferase Genetic Polymorphism

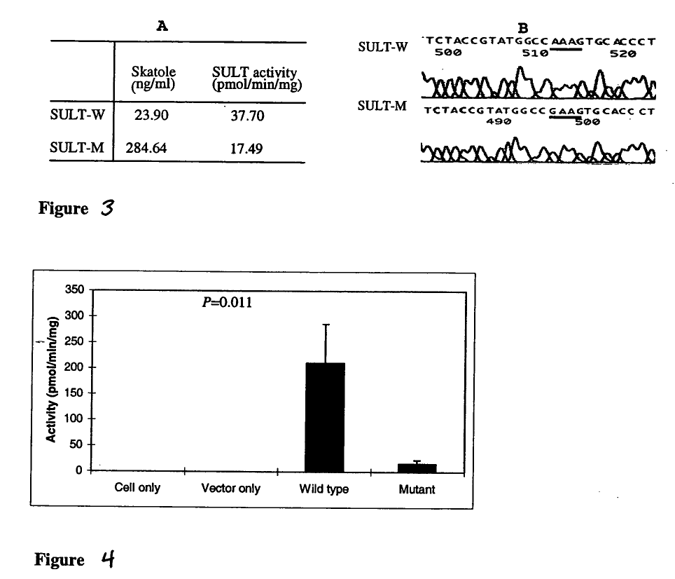

In order to identify any genetic polymorphism of phenol sulfotransferase that may alter the metabolic capacities of the enzyme, a polymerase chain reaction technique combined with single strand conformational polymorphism (PCR-SSCP) was used to scan the phenol sulfotransferase coding region from porcine liver tissues. The phenol sulfotransferase full-length cDNA was amplified by PCR with the primer pair: forward primer 5′ ATG GAG CCG GTC CAG GAC A 3′; reverse primers: 5′ TCA CAG CTC AGA GCG GAA GC 3′ . The resulting PCR products were about 900 bp in size and were digested with KpnI and subjected to SSCP analysis using our optimized system. We found that there are several different polymorphisms present in the phenol sulfotransferase coding region (data not shown). One substitution (FIG. 3-B) of Lys¹⁴⁷ (AAA) to Glu¹⁴⁷ (GAA) at nucleotide 546 bp was of particular interest because of the big difference in the skatole level between wild type and mutant samples (FIG. 3-A). We proposed that the substitution might result in decreased phenol sulfotransferase activity for this individual and that the skatole level would be higher due to decreased activity of this enzyme important in clearing skatole from the body.

To evaluate the above hypothesis and investigate the association of this genetic polymorphism to phenol sulfotransferase activity, recombinant DNA containing the substitute mutant (A→G) and wild type of pig phenol sulfotransferase cDNA were used to transfect mammalian cells, the activities of recombinant proteins produced were assayed using ρ-nitrophenol as a substrate (FIG. 4). For the wild type, sulfation activity was 211.24±75.57 pmol/min/mg, whereas for the Lys¹⁴⁷ to Glu¹⁴⁷ mutation, the activity was 15.97±7.18 pmol/min/mg, showing a significant difference between the mutant and wild type (P<0.05). This result indicates that Lys¹⁴⁷ is crucial for the catalytic activity of phenol sulfotransferase. The results strongly support our suggestion that the Lys¹⁴⁷ to Glu¹⁴⁷ mutation caused a decrease in the catalytic activity of phenol sulfotransferase and hence result in a higher level skatole in the pig.

Phenol sulfotransferase genes have been extensively investigated in humans. In pigs, it has been reported that phenol sulfotransferase is negatively correlated with skatole accumulation in fat (Babol et al, 1998; Diaz and Squires, 2003). However, the information about the phenol sulfotransferase gene, its expression in different tissues and how a genetic variant of it affects sulfation activity, hence skatole level in pig has not been previously reported. In humans, three members of the phenol sulfotransferase family, SULT1A1, SULT1A2, and SULT1A3 have been cloned and characterized. DNA sequences and the structure of these three enzymes are highly homologous, and all three genes are localized on chromosome 16p12.1 (Dooley et al, 1993; Gaedigk et al, 1997; Aksoy et al, 1994). Both SULT1A1 and SULT1A2 catalyze the sulfation of ρ-nitrophenol (Raftogianis et al, 1997), while SULT1A3 shows a trivial activity for ρ-nitrophenol (Veronese et al, 1994). Therefore, SULT1A1 and SULT12A are considered the main enzymes that catalyze sulfation in humans. We designed the first primer pair based on human SULT1A1 and SULT1A2 cDNA sequences. Therefore, by using the designed primers, we screened out the first fragment, and subsequently the whole sequence of pig phenol sulfotransferase cDNA. To further character this gene, this pig putative phenol sulfotransferase cDNA was subcloned into the expression vector and used to transfect COS-7 cells. The expressed enzyme showed high catalytic activity towards the ρ-nitrophenol substrate. The results demonstrate that this cDNA is indeed pig phenol sulfotransferase, and is one of isoforms of SULT1A1 or SULT1A2 rather than SULT1A3. In humans, SULT1A1 has up to 10-fold higher phenol sulfotransferase activity compared with that of SULT1A2 (Raftogianis et al, 1997). It is also suggested that SULT1A2 does not contribute substantially to the sulfation of endogenous or xenobiotic agents in vivo (Dooley, 1998). Due to the high identity (96%) between human SULT1A1 and SULT1A2 cDNAs, the pig phenol sulfotransferase cDNA and its deduced amino acid sequence showed the same homology (86%) with human SULT1A1 and SULT1A2 cDNA and amino acid sequences. SULT1A1 and SULT1A2 genes in human have been mapped to chromosome 16p12.1. When we searched against the human genomic database with the pig phenol sulfotransferase cDNA sequence, we found that this cDNA hit a human genomic clone (NT_{—}010393.13), which contains both SULT1A1 and SULT1A2 from chromosome 16p12.1. The hit scores showed that pig cDNA sequence has 91% identity with human SULT1A1 and 88% identity with human SULT1A2. All these finding taken together suggest that the cDNA we isolated from pig liver is SULT1A1.

Applicants isolated pig phenol sulfotransferase from liver tissue using the RACE method, then performed PCR-SSCP analysis to scan its coding region. A substitution from A to G at nucleotide 546 bp, which caused a change in amino acid sequence from Lys¹⁴⁷ to Glu¹⁴⁷ was identified. To help clarify possible genotype-phenotype correlation for the genetic mutation, we next determined the sulfation activity of the protein encoded by SULT1A1 and SULT1A1 Lys¹⁴⁷ to Glu⁴⁷ mutant expressed in COS-7 cells. The result showed that the transition from A to G significantly reduced enzymatic activity.

References

Aksoy I A, Callen D F, Apostolou, S, Her C, Weinshilboum R M (1994) Thermolabile phenol sulfotransferase gene (STM): Localization to human chromosome 16p11.2. Genomics 23, 275-277

Babol J, Squires E J, Lundstrom K (1998) Relationship between oxidation and conjugation metabolism of skatole in pig liver and concentrations of skatole in fat. J. Anim. Sci. 76, 829-838

Bamber D E, Fryer A A, Strange, R C, Elder J B, Deakin M, Rajagopal R, Fawole A, Gilissen R, Campbell F C, Coughtrie W H (2001) Phenol sulphotransferase SUL1A1*1 genotype is associated with reduced risk of colorectal cancer. Pharmacogenetics 11, 679-685

Chen G, Rabjohn P A, York J L, Wooldridge C, Zhang D, Falany C N, Radominska-Pandya A (2000) Carboxyl Residues in the active site of human phenol sulfotransferase (SULT1A1). Biochemistry 39, 16000-16007

Diaz, G. J. and Squires, E. J. (2000). Metabolism of 3-Methylindole by Porcine Liver Microsomes: Responsible Cytochrome P450 Enzyme. Toxicological Science 55, 284-292.

Diaz, G J and Squires E J (2003) Phase II in vitro metabolism of 3-methylindole metabolites in porcine liver. Xenobiotica 33, 485-498.

Dooley T P (1998) Molecular biology of the human phenol sulferasferase gene family. The Journal of experimental zoology 282, 223-230

Dooley T P, Obermoeller R D, Leiter E H, Chapman H D, Falany C N, Deng Z, Siciliano M J (1993) Mapping of the phenol sulfotransferase gene (STP) to human chromosome 16p12.1-p11.2 and to mouse chromosome 7. Genomics 18, 440-443

Henry T, Kliewer B, Palmatier R, Ulphani J S, Beckmann J D (1996) Isolation and characterization of a bovine gene encoding phenol sulfotransferase. Gene 174, 221-224

Her C, Raftogianis R, Weinshilboum R M (1996) Human phenol sulfotransferase STP2 gene: Molecular cloning, structural characterization, and chromosome localization. Genomics 33, 409-420

Honma W, Kamiyama Y, Yoshinari K, Sasano H, Shimada M, Nagata K, Yamazoe Y (2001) Enzymatic characterization and interspecies difference of phenol sulfotransferases, ST1A forms. Drug Metabolism and Disposition 29, 274-281

Gaedigk A, Beatty B G, Grant D M (1997) Cloning, structural organization, and chromosomal mapping of the human phenol sulfotransferase STP2 gene. Genomics 40, 242-246

Raftogianis R B, Wood T C, Otterness W D, Loon J A, Weinshilboum R M (1997) Phenol sulfotransferase pharmacogenetics in human: association of common SULT1A1 alleles with TS PST phenotype. Biochemical and Biophysical Research Communications 239, 298-304

Sakakibara Y, Yanagisawa K, Takami Y, Nakayama T, Suiko M, Liu M C (1998) Molecular cloning, expression, and functional characterization of novel mouse sulfotransferases. Biochemical and Biophysical research communications 247, 681-686

Seth P, Lunetta K L, Bell D W et al (2000) Phenol sulfotransferases: Hormonal regulation, polymorphism, and age of onset of breast cancer. Cancer Research 60, 6859-6863

Veronese M E, Burgess W, Zhu X, McManus M E (1994) Functional characterization of two human sulphotransferase cDNAs that encode monoamine- and phenol-sulphating forms oh phenol sulfotransferase: substrate kinetics, thermal-stability and inhibitor-sensitivity studies. Bichemical Journal 302, 497-502

Wang Y, Spitz M R, Tsou A M, Zhang K, Makan N, Wu X (2002) Sulfotransferase (SULT) 1A1 polymorphism as a predisposition factor for lung cancer: a case-control analysis. Lung Cancer 35, 137-142

Weinshilboum R M, Otterness D M, Aksoy I A, Wood T C, Her C, Raftogianis R B (1997) Sulfation and sulfotransferases 1: Sulforansferase molecular biology: cDNAs and genes. The FASEB Journal 11, 3-14

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

1. A method of genetically typing animals to determine those with desired boar taint characteristics, comprising:
obtaining a sample of genetic material from said animal; and
assaying for the presence of a sulfotransferase allele characterized by the following:
a) a polymorphism in a sulfotransferase gene, said polymorphism being one which characterizes a first allele and a second allele which differ in activity of the sulfotransferase enzyme.

2. The method of claim 1 wherein said polymorphism is a polymorphism at nucleotide position 546 of SEQ ID NO:5.

3. The method of claim 1 wherein said polymorphism is a lys to glu substitution at amino acid 147 of the sulfotransferase enzyme.

4. The method of claim 3 wherein said glu substitution results in a decrease of activity of the sulfotransferase enzyme.

7. The method of claim 11 wherein said step of assaying is selected from the group consisting of: restriction fragment length polymorphism (RFLP) analysis, minisequencing, MALD-TOF, SINE, heteroduplex analysis, one base extension methods, single strand conformational polymorphism (SSCP), denaturing gradient gel electrophoresis (DGGE) and temperature gradient gel electrophoresis (TGGE).

8. A method of genetically typing animals according to skatole metabolism comprising:
obtaining a sample of genetic material from an animal;
assaying for the presence of an allele characterized by a polymorphism in a sulfotransferase gene present in said sample, and
correlating said allele with skatole metabolism and concomitant boar taint in said animal and typing animals based upon the presence of said allele and boar taint.

9. The method of claim 8 wherein said polymorphism results in a substitution at position 546 of SEQ ID NO:5.

10. The method of claim 8 wherein said step of assaying is selected from the group consisting of: restriction fragment length polymorphism (RFLP) analysis, minisequencing, MALD-TOF, SINE, heteroduplex analysis, one base extension methods, single strand conformational polymorphism (SSCP), denaturing gradient gel electrophoresis (DGGE) and temperature gradient gel electrophoresis (TGGE).

11. The method of claim 9 further comprising the step of amplifying the amount of sulfotransferase gene or a portion thereof which contains said polymorphism.

12. A method of determining genetic variability in animals which is linked to skatole metabolism comprising:
obtaining a biological sample from a group, line, population or family of animals, said sample comprising a nucleotide sequence encoding a sulfortransferase enzyme;
comparing said sequence to a reference sequence to identify a polymorphism;
correlating said polymorphism with variability in skatole metabolism for said group, line, population or family of animals.

13. A method of screening animals to determine those with desired boar taint characteristics, comprising:
obtaining a sample of genetic material from said animal; and
assaying for the presence of a genotype in said animal which is associated with improved boar taint, said genotype characterized by the following:
a) a polymorphism in a sulfotransferase gene, said polymorphism being one which is associated with improved boar taint characteristics.

14. A nucleotide sequence which encodes a sulfotransferase protein, having a substitution of an A to G substitution at position 546 of SEQ ID NO:5 or its equivalent as determined by BLAST, said nucleotide sequence comprising one or more of the following:
(a) SEQ ID NO:5;
(b) a sequence which will hybridize under conditions of high stringency to the sequences in (a); or
(c) a sequence with at least about 90% sequence identity to the sequences in (a).

15. A porcine nucleotide sequence which encodes a sulfotransferase protein said nucleotide sequence comprising one or more of the following:
(a) SEQ ID NO:5;
(b) a sequence which will hybridize under conditions of high stringency to the sequences in (a); or
(c) a sequence with at least about 90% sequence identity to the sequences in (a).

16. A nucleotide sequence which encodes a sulfotransferase protein, protein characterized by one the following:
(a) SEQ ID NO:5;
(b) a conservatively modified variant of the sequences in (a); or
(c) a sequence with at least about 90% sequence identity to the sequences in (a).

17. A sulfotransferase protein according to claim 16.

18. A sulfotransferase protein, said protein comprising an amino acid sequence comprising one of the following:
(a) SEQ ID NO:6;
(b) conservatively modified variant of (a); or
(c) a sequence with at least about 80% homology to a sequence in (a).

19. A nucleotide sequence encoding the protein of claim 18.


SEQUENCE LISTING (14 sequences)

SEQ ID NO: 1 - protein, 295 aa, porcine
MEPVQDTYRPPLEYVKGVPLIKYFAEALGPLESFQAWPDDVLISTYPKSGTTWVSEILDLIYQGGDLQKCQRAPIFVRVPFLEFKIPRCPTGFELLKDTPAPRLLKTHLPLTLLPQTLLDQKVKVVYVARNAKDVAVSYYHFYRMAKVYPNPGTWDSFLEDFMAGEVSYGSWYQHVQEWWELRHTHPVLYLFYEDMKENPKREIQKILEFVGRSLPEETVEDIVQHTSFQEMKNNAMTNYRTLPSDLLDHSISAFMRKGITGDWKSTFTVAQNERFEADYAEKMAGCNLRFRSEL

SEQ ID NO: 2 - protein, 295 aa, Homo sapiens
MELIQDTSRPPLEYVKGVPLIKYFAEALGPLQSFQARPDDLLISTYPKSGTTWVSQILDMIYQGGDLEKCHRAPIFMRVPFLEFKAPGIPSGMETLKDTPAPRLLKTHLPLALLPQTLLDQKVKVVYVARNAKDVAVSYYHFYHMAKVHPEPGTWDSFLEKFMVGEVSYGSWYQHVQEWWELSRTHPVLYLFYEDMKENPKREIQKILEFVGHSLPEETVDFMVQHTSFKEMKKNPMTNYTTVPQEFMDHSISPFMRKGMAGDWKTTFTVAQNERFDADYAEKMAGCSLSFRSEL

SEQ ID NO: 3 - protein, 295 aa, Homo sapiens [sequence omitted]

SEQ ID NO: 4 - protein, 295 aa, Homo sapiens
MELIQDTSRPPLEYVKGVPLIKYFAEALGPLQSFQARPDDLLINTYPKSGTTWVSQILDMIYQGGDLEKCNRAPIYVRVPFLEVNDPGEPSGLETLKDTPPPRLIKSHLPLALLPQTLLDQKVKVVYVARNPKDVAVSYYHFHRMEKAHPEPGTWDSFLEKFMAGEVSYGSWYQHVQEWWELSRTHPVLYLFYEDMKENPKREIQKILEFVGRSLPEETMDFMVQHTSFKEMKKNPMTNYTTVPQELMDHSISPFMRKGMAGDWKTTFTVAQNERFDADYAEKMAGCSLSFRSEL

SEQ ID NO: 5 - DNA, 1201 bp, porcine
  CDS (108)..(1178)
acgcggggagtgcagtgaccacaccataccccactttaagccacaggacacagaggtgtcacaagccaggatccagagagggcaggggaggagagcgagctcgcagcatggagccggtccaggacacctaccgcccgccactggagtacgtgaagggggtccctctcatcaagtactttgcagaggcactggggccactggagagtttccaagcttggcccgatgacgtgctgatcagcacctatcccaaatctggtaccacctgggtgagcgagatcctggacctaatctaccagggtggtgacctgcagaagtgtcagagagcccccatctttgtgcgggtgcccttccttgagttcaaaatccccaggtgtcccacaggttttgagctgctgaaagatacaccagccccacggctcctcaagacacacttgcccctgaccctgctaccccagactctgctggaccagaaggtcaaggtggtctacgtcgcccgcaacgcaaaggacgtggccgtctcctattaccacttctaccgtatggccaaagtgtaccctaaccctggcacgtgggacagcttcctggaagacttcatggccggagaagtgtcctacgggtcctggtaccagcacgtgcaggagtggtgggagctgcgtcacacccaccctgtcctctatctcttctacgaggacatgaaggagaaccccaagagggagattcagaagatcctggagtttgtggggcgctcgctgccagaggagacggtggaggacatcgtccagcacacgtccttccaggagatgaagaacaacgccatgaccaactaccgcaccctcccctccgacctcctggaccacagcatctcagccttcatgaggaaaggcatcactggagattggaaaagcaccttcacagtggcccagaacgagcgctttgaggccgactatgctgagaagatggcaggctgtaacctccgcttccgctctgagctgtgagtggtggcgttcacggaggaggggtgtgaagcaagcccggctggagcctcaaggataaaatctgggagtttcctggcggctcagcggggtaaagatcatgtgttgtcactgctgtgtggcctataaacttctgcatgctgttgctgtggcccaaaaaggtgaataaatcaaatctgatgtgtgttcaaaaaaaaaaaaaaaaaaaa

SEQ ID NO: 6 - protein, 295 aa, porcine; identical to SEQ ID NO: 1

SEQ ID NO: 7 - protein, 41 aa, porcine
VVAFTEEGCEASPAGASRIKSGSFLAAQRGKDHVLSLLCGL

SEQ ID NO: 8 - protein, 11 aa, porcine
TSACCCCGPKR

SEQ ID NO: 9 - protein, 4 aa, porcine
INQI

SEQ ID NO: 10 - DNA, 30 bp, porcine
aagcagtggtatcaacgcagagtacgcggg

SEQ ID NO: 11 - DNA, 19 bp, Homo sapiens
cacagctcagagcggaagc

SEQ ID NO: 12 - DNA, 22 bp, Homo sapiens
agtggtgggagctgcgtcacac

SEQ ID NO: 13 - DNA, 19 bp, porcine
atggagccggtccaggaca

SEQ ID NO: 14 - DNA, 20 bp, porcine
tcacagctcagagcggaagc